(19) United States
(12) Patent Application Publication
(10) Pub. No.: US 2015/0065800 A1
(43) Pub. Date: Mar. 5, 2015

(54) VIDEO ENDOSCOPE

(71) Applicant: OLYMPUS WINTER & IBE GMBH, Hamburg (DE)

(72) Inventors: Sebastian JUNGBAUER, Hamburg (DE); Patrick SCHERR, Alt-Moelln (DE); Sebastian STUEHLE, Hamburg (DE); Sven PABST, Giekau (DE)

(73) Assignee: OLYMPUS WINTER & IBE GMBH, Hamburg (DE)

(21) Appl. No.: 14/538,148

(22) Filed: Nov. 11, 2014

Related U.S. Application Data
(63) Continuation of application No. PCT/EP2013/001368, filed on May 8, 2013.

(30) Foreign Application Priority Data
May 18, 2012 (DE) 102012208358.8

(51) Int. Cl. A61B 1/00; A61B 1/05

(52) U.S. Cl. CPC A61B 1/00126; A61B 1/051; A61B 1/00165; A61B 1/0011; A61B 1/00124; A61B 1/053; A61B 1/00096; A61B 1/00142
USPC 600/110; 600/109; 600/112

(57) ABSTRACT

A video endoscope including: an elongated housing; an image sensor unit arranged in the elongated housing; an optical transmitting unit arranged in the elongated housing and connected to the image sensor unit; and a through contact device, wherein the optical transmitting unit is arranged at a proximal end of the elongated housing on the through-contact device, the through-contact device penetrates through a wall of the elongated housing and the through-contact device is arranged such that it is mounted in a hermetically sealed manner in the wall of the elongated housing.

### CROSS-REFERENCE TO RELATED APPLICATION

The present application is a continuation of PCT/EP2013/001368 filed on May 8, 2013, which is based upon and claims the benefit to DE 10 2012 208 358.8 filed on May 18, 2012, the entire contents of each of which are incorporated herein by reference.

### BACKGROUND

1. Field

The present application relates to a video endoscope comprising an elongated, preferably hermetically sealed, housing for accommodating an image sensor unit, more particularly video camera, preferably arranged in the distal region of the housing, wherein an optical transmitting unit connected to the image sensor unit is arranged in the housing.

2. Prior Art

Video endoscopes are designed elongated with a small cross-section. In generic video endoscopes, image sensors and the upstream optical systems are normally arranged in the distal region of the endoscope shaft, that is, in the region of the endoscope tip. Image signals and control signals are transmitted via signal lines to or respectively from the proximal end, that is, the handle.

The autoclavability of the endoscope is an important requirement. During autoclaving, the endoscope is treated with hot steam under high pressure. In the case of optical endoscopes and in particular video endoscopes, it is necessary to protect the optical components and the image sensor from steam which otherwise condenses on the lens system upon cooling and can impair the optical quality of the system. Video endoscopes are therefore normally constructed in a hermetically sealed manner. The hermetic seal prevents steam from penetrating into the hermetically sealed region. With conventional video optical systems, this normally extends from the shaft tip into the handle.

Video endoscopes are generally designed elongated and with a small cross-section due to their application, for example, for introduction into the body channels of a patient. Corresponding video endoscopes transmit video signals from the distally arranged video camera via electrical lines to a proximal end of the video endoscope, and the video signals are conducted to the outside through its housing with a contacting device. Between the contacting device and the video camera, the conductors or respectively lines run on an insulated conductor support.

DE 10 2004 023 866 B3 shows a conductor support in the form of conductor plates both in a flexible and in a rigid design. In the case of the rigid design, the conductor support can also be used to transmit forces, e.g. torsional forces, in order for example to turn the video camera from the proximal end of the video endoscope or to shift it longitudinally for focusing. The conductor plate can also be designed in the form of a triangular or round tube.

In video technology, data quantity is increasing due to continuously higher resolution technologies. In particular, the number of conductor paths and/or the frequency of the data transmission are thus also increasing.

With video endoscopes, an optical system, for example an objective looking straight ahead or laterally, to which an image sensor or a pair of image sensors is connected, which convert the received light into electronic image information and forward it on proximally as electronic signals, is normally located on the distal tip of an endoscope shaft. Pairs of image sensors can be used for example for stereo video endoscopes for producing a spatial impression, for improving the color rendering, or for setting different sensitivities or different analyses, for which different optical properties are needed.

The electrical lines, with which the electrical signals inside the endoscope shaft are forwarded, can be cables with several shielded and unshielded wires, flexible conductor plates or the like.

In video endoscopes made by Olympus Winter & Ibe GmbH, Hamburg, both the optical system and the image sensor are located in a hermetically sealed space. A hermetically sealed through-contact of these electrical lines is thus present. With corresponding video endoscopes, the hermetically sealed through-contact takes place by means of metal pins or respectively metallic contact tags poured into glass. The electrical lines are soldered directly to the metal pins.

Furthermore, video endoscopes are known in the state of the art, which have in the hermetic space an optical transmitting unit for transmitting the image data received by the image sensor.

### SUMMARY

An object is to provide a surgical instrument, such as video endoscopes, with a stable arrangement of an optical transmitting device for transmitting image data from the hermetically encapsulated housing, wherein the effort and the installation should be as minimal as possible.

This object is solved through a video endoscope comprising an elongated, which can be hermetically sealed, housing for accommodating an image sensor unit, more particularly video camera, which can be arranged in the distal region of the housing, wherein an optical transmitting unit connected to the image sensor unit is arranged in the housing, which is further characterized by the fact that the optical transmitting unit is arranged at the proximal end of the housing, which can be in the end region at a proximal end face of the housing, on a through-contact device, wherein the through-contact device penetrates through a wall of the housing and the through-contact device is arranged such that it is mounted in a hermetically sealed manner in the wall of the housing.

The present application is based on the idea of providing a holder for the optical transmitting device by means of the rigid i.e. inflexible conductor plate in the wall of the hermetically sealed video endoscope housing, wherein a hermetical seal of the housing is achieved by means of the conductor plate penetrating the housing wall and arranged in the wall and electrical connections are simultaneously designed or respectively provided between devices outside of the housing with devices inside the housing via corresponding conductor paths of the conductor plate.

A further embodiment of the video endoscope is characterized in that the through-contact device is mounted on the proximal end face of the housing in a plate, in particular cover plate for the housing, provided on the proximal end of the housing.

For this, it is advantageously provided in the case of the video endoscope that the through-contact device for the optical transmitting unit is arranged in the wall of the housing such that an inner part of the through-contact device protrudes into the inside of the housing and a second, outer part of the through-contact device on the outside of the housing protrudes outwardly. The conductor paths of the conductor plate present in the housing and outside the housing are hereby connected with the corresponding lines.

In particular, the through-contact device is designed as a conductor plate with electrical lines, wherein in particular the conductor plate has a multi-layer ceramic with electric or electrically conductive lines, wherein the electrical or electrically conductive lines are or will be produced by means of the high-temperature multi-layer ceramic method (HTCC method).

Moreover, it is suggested in an exemplary embodiment of the video endoscope that one or more image data transmission lines connected with the image sensor unit are arranged in the housing, wherein the one or more image data transmission lines are connected on the proximal end of the housing, which can be in the end region to a proximal end face of the housing, with a through-contact device and/or the optical transmitting unit.

One or more image data transmission lines connected with the image sensor unit which can be arranged in the housing designed as an endoscope shaft, wherein the one or more image data transmission lines for transmitting image data are connected on the proximal end of the housing, which can be in the end region to a proximal end face of the housing, with the optical transmitting unit.

In particular, a socket is provided on the proximal end of the housing, wherein the optical transmitting unit is arranged on one end of the socket and a light wave conductor surrounded by a ferrule is arranged on the other end of the socket such that the optical transmitting unit is arranged opposite the end of the light wave conductor.

Moreover, it is advantageous that a fiber-optic data transmission from the hermetically sealed region of the housing or respectively the endoscope shaft is designed by providing a socket on the proximal end face or respectively front side of the housing, wherein the image data is transmitted out of the hermetic housing by means of the optical transmitting unit on the inner end of the socket and the optical signals are received by the light wave conductor arranged on the other, outer end of the socket and are transmitted accordingly to an image representation unit or the like. It is also hereby possible to provide a higher data transmission rate.

In particular, a light wave conductor is understood as an optical fiber, such as a glass fiber, or a bundle of optical fibers, e.g. fiber glass bundles, wherein the light wave conductor is provided with a ferrule on one end, which is arranged or is arrangeable on the socket of the housing of the video endoscope. Moreover, the individual fibers or the bundle of fibers is protected by a sleeve wherein the ferrule is attached on the end in the region of the end face of the glass fiber(s).

The ferrule can be glued on or applied to the fibers or glass fibers in a melting process. A high water or respectively steam impermeability is hereby achieved using suitable adhesives or through the melting of the ferrule with the glass fibers.

Moreover, the video endoscope is characterized in one embodiment in that a glass is arranged between the ends of the socket, in particular between the optical transmitting unit inside the housing and the end of the light wave conductor arranged on the socket, wherein the glass can be soldered into the socket. A hermetic seal is hereby achieved, wherein an optical transmission between the transmitting unit and the light wave conductor is simultaneously enabled.

Moreover, the optical transmitting unit can be soldered to the socket, whereby the optical transmitting unit is connected with the socket in a steam-impermeable manner.

Furthermore, the ferrule for the light wave conductor can be soldered to the socket. The end face of the light wave conductor or respectively the glass fibers is also hereby sealed in a steam-impermeable manner.

Moreover, it is provided in an alternative embodiment that the ferrule is arranged in a bushing such that the ferrule is surrounded by the bushing and is received in it, wherein the bushing is arrangeable or arranged, in particular attachable or attached, to the end of the socket opposite the optical transmitting unit.

Furthermore, one embodiment of the video endoscope is characterized in that the socket is arranged in the or a plate, in particular cover plate or sealing plate, provided on the proximal end of the housing, on the proximal end face of the housing. The (end) plate provided on the proximal end of the housing can thereby made of glass or metal.

Moreover, it is provided in a further embodiment of the video endoscope that the through-contact device is arranged on the proximal end face of the housing, which can be next to the socket, wherein the through-contact device is connected with the image sensor unit, in particular video camera, arranged on the distal end of the housing via electrical conductors. It is hereby possible for example to provide a proximal hermetic duct for electrical lines by means of a high-temperature multi-layer ceramic (HTCC), wherein it is possible to charge the electrical or electro-optical components automatically. Moreover, a component is also produced using the HTCC (High Temperature Cofired Ceramics) technology that has electrical and optical through-contact devices.

Further characteristics will become apparent from the description of embodiments together with the claims and the included drawings. Such embodiments can fulfil individual characteristics or a combination of several characteristics.

### BRIEF DESCRIPTION OF THE DRAWINGS

The present application is described below, without restricting the general idea of the present application, using exemplary embodiments with reference to the drawings, whereby reference is expressly made to the drawings with regard to all details according to the present application that are not explained in greater detail in the text. The figures show:

FIG. 1 schematically illustrates a cross-section through the proximal region of a video endoscope according to and embodiment; and

FIG. 2 schematically illustrates a cross-section through the proximal region of a video endoscope according to another embodiment.

### DETAILED DESCRIPTION

In the drawings, the same or similar types of elements and/or parts are provided with the same reference numbers so that a re-introduction is omitted.

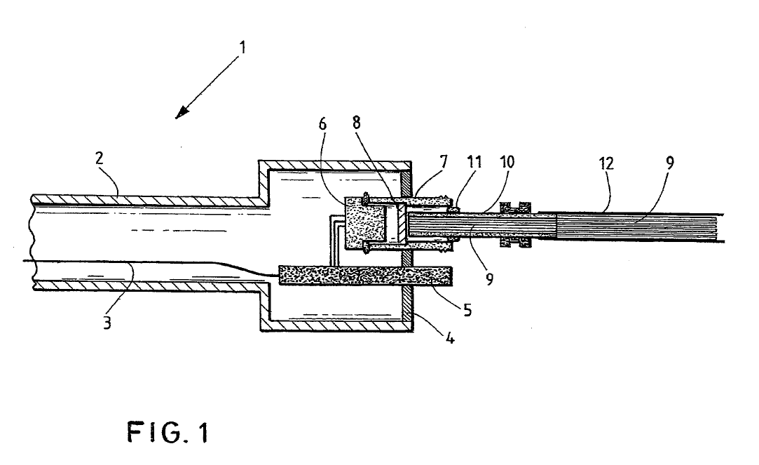

FIG. 1 shows schematically in cross-section a proximal end region of a video endoscope 1 shown schematically. The video endoscope 1 has an endoscope shaft 2 elongated as a housing, on the distal end of which a window is provided, wherein a video camera is arranged as an optical image sensor unit behind the window in the endoscope shaft. In order to transmit the image data of the video camera, an image data line 3 is provided in the endoscope shaft 2 in order transmit the image data of the video camera.

An end plate 4, e.g. made of glass, is arranged in the shown proximal end region of the endoscope shaft 2, in order to close the proximal end face of the endoscope shaft 2. A through-contact device 5 designed as a rigid HTCC conductor plate is arranged in the end plate 4 in order to provide electrical power for the units or devices such as the video camera inside the endoscope shaft 2 via corresponding connections or respectively electrical lines. The through-contact device 5 thereby penetrates the end plate 4, wherein the through-contact device 5 is arranged in a hermetically sealed manner in the end plate 4.

In an alternative embodiment, a plate made of metal is provided as a wall of the housing instead of the end plate 4 made of glass in the proximal end region, which is penetrated by the through-contact device 5 designed as conductor plate and is arranged in it in a hermetically sealed manner, wherein in particular the electrical conductor paths of the conductor plate are electrically insulated with respect to the end plate 4 made of metal.

Moreover, the end plate 4 is soldered on the border to the endoscope shaft 2 for example made of metal, whereby a hermetically sealed housing results that is not only liquid-impermeable but also steam-impermeable. Furthermore, the optical and electronic components are also thereby appropriately protected in the case of a hot steam sterilization of the video endoscope.

The through-contact device 5 is designed for example as a conductor plate using the HTCC technology (High Temperature Cofired Ceramics) with several layers of ceramic and is hermetically soldered in the end plate 4. Inside the endoscope shaft 2, the through-contact device 5 has electrical or electronic components as well as an optocoupler (not shown here) for the transmitting unit 6. Furthermore, the image data line 3 is connected with an electro-optical transmitting unit 6 by means of the through-contact device 5, in order to transmit optical signals by means of the electro-optical transmitting unit 6 through conversion of the image data captured by the video camera.

Furthermore, a socket 7, which penetrates the end plate 4 designed e.g. as a glass plate, is provided in the proximal end region of the video endoscope 1. A glass body 8 is arranged inside the socket 7, wherein in particular the glass body 8 is soldered inside the socket 7. Furthermore, the socket 7 is also soldered to the end plate 4 on the outer perimeter.

The electro-optical transmitting unit 6 is arranged inside the endoscope shaft 2 on the end of the socket 7 protruding into the endoscope shaft 2, wherein in particular the optical transmitting unit 6 is soldered on the end of the socket 7 and is connected with the socket 7.

Inside the socket 7, the end of a light wave conductor 9 is arranged on the other end or respectively outer end of the socket 7 in order to receive the light signals send by the optical transmitting unit 6. The end of the light wave conductor 9 is thereby surrounded by a ferrule 10, wherein the ferrule 10 is connected to the socket 7 by means of a soldering 11. The ferrule 10 is glued to the end of the light shaft conductor 9 through suitable selection of adhesives or is connected in a steam- or water-impermeable manner with the glass fibers as a result of a melting process.

The signals received from the image data line 3 are transmitted to an image representation device by means of the light wave conductor 9. Furthermore, the light wave conductor 9 is surrounded on the end facing away from the ferrule 10 by a corresponding protective sleeve 12.

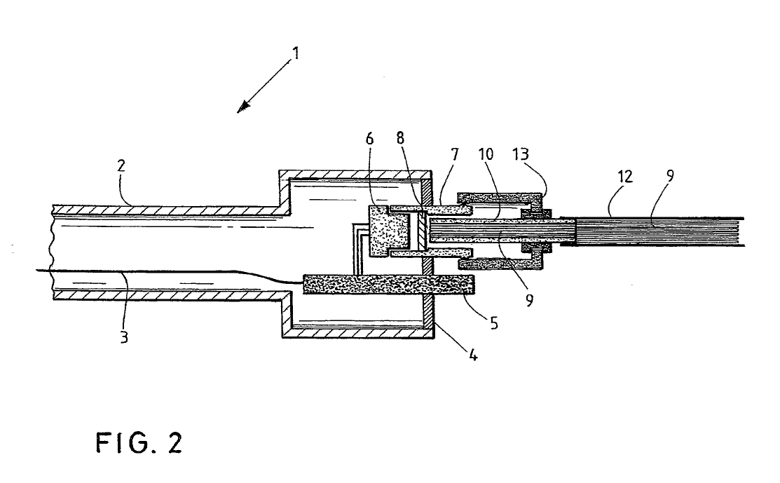

FIG. 2 shows schematically another exemplary embodiment for the arrangement of an optical transmitting unit 6 in the proximal end region of the video endoscope 1. Compared to the exemplary embodiment in FIG. 1, the ferrule 10 is surrounded by a bushing housing 13 according to the embodiment in FIG. 2 such that the bushing housing 13 is releasably fitted on the outer socket end of the socket 7. A type of plug connection is hereby designed for the light wave conductor 9 so that that the light wave conductor is releasably connectible with the socket 7.

All named characteristics, including those taken from the drawings alone and individual characteristics, which are disclosed in combination with other characteristics, are considered alone and in combination as essential for the present application. Embodiments according to the present application can be realized by individual features, or a combination of several features.

### LIST OF REFERENCE NUMBERS

1 Video endoscope
2 Endoscope shaft
3 Image data line
4 End plate
5 Contact device
6 Optical transmitting unit
7 Socket
8 Glass body
9 Light wave conductor
10 Ferrule
11 Soldering
12 Protective sleeve
13 Bushing housing

What is claimed is:

1. A video endoscope comprising:
an elongated housing;
an image sensor unit arranged in the elongated housing;
an optical transmitting unit arranged in the elongated housing and connected to the image sensor unit; and
a through contact device, wherein the optical transmitting unit is arranged at a proximal end of the elongated housing on the through-contact device, the through-contact device penetrates through a wall of the elongated housing and the through-contact device is arranged such that it is mounted in a hermetically sealed manner in the wall of the elongated housing.

2. The video endoscope according to claim 1, wherein the elongated housing is hermetically sealed.

3. The video endoscope according to claim 1, wherein the image sensor unit is a video camera.

4. The video endoscope according to claim 1, wherein the image sensor unit is arranged in a distal region of the elongated housing.

5. The video endoscope according to claim 1, wherein the optical transmitting unit is arranged at an end region at a proximal end face of the elongated housing.

6. The video endoscope according to claim 1, wherein the through-contact device is mounted on the proximal end face of the elongated housing in a plate provided on the proximal end of the elongated housing.

7. The video endoscope according to claim 6, wherein the plate is a cover plate for the elongated housing.

8. The video endoscope according to claim 1, wherein the through-contact device for the optical transmitting unit is arranged in the wall of the elongated housing such that an inner part of the through-contact device protrudes into an inside of the elongated housing and a second, outer part of the through-contact device on an outside of the elongated housing projects outwardly.

9. The video endoscope according to claim 1, wherein the through-contact device is configured as a conductor plate with electrical lines.

10. The video endoscope according to claim 9, wherein the conductor plate has a multi-layer ceramic with electric or electrically conductive lines.

11. The video endoscope according to claim 10, wherein the electrical or electrically conductive lines are formed by means of a high-temperature multi-layer ceramic method.

12. The video endoscope according to claim 1, further comprising one or more image data transmission lines connected with the image sensor unit and arranged in the elongated housing, wherein the one or more image data transmission lines are connected on the proximal end of the elongated housing with one or more of the through-contact device and the optical transmitting unit.

13. The video endoscope according to claim 12, wherein the one or more image data transmission lines are connected in an end region to a proximal end face of the elongated housing.

14. The video endoscope according to claim 1, further comprising a socket provided on the proximal end of the elongated housing, wherein the optical transmitting unit is arranged on one end of the socket and a light wave conductor surrounded by a ferrule is arranged on an other end of the socket such that the optical transmitting unit is arranged opposite an end of the light wave conductor.

15. The video endoscope according to claim 14, further comprising a glass arranged between the one end and the other end of the socket.

16. The video endoscope according to claim 15, wherein the glass is arranged between the optical transmitting unit and the end of the light wave conductor arranged on the socket.

17. The video endoscope according to claim 15, wherein the glass is soldered into the socket.

18. The video endoscope according to claim 14, wherein the optical transmitting unit is soldered to the socket.

19. The video endoscope according to claim 14, wherein the ferrule for the light wave conductor is soldered to the socket.

20. The video endoscope according to claim 14, wherein the ferrule is arranged in a bushing such that the ferrule is surrounded by the bushing, wherein the bushing is arranged to the end of the socket opposite the optical transmitting unit.

21. The video endoscope according to claim 14, wherein the ferrule is attached to the end of the socket opposite the optical transmitting unit.

22. The video endoscope according to claim 14, wherein the through-contact device is mounted on the proximal end face of the elongated housing in a plate provided on the proximal end of the elongated housing and the socket is arranged in or on the plate.

23. The video endoscope according to claim 1, wherein the through-contact device is connected with the image sensor unit arranged on the distal end of the elongated housing via electrical conductors.
